(19) United States
(12) Patent Application Publication
(10) Pub. No.: US 2004/0234488 A1
(43) Pub. Date: Nov. 25, 2004

(54) Personal cleaning composition

(76) Inventors: Craig Joseph Davies, Staffs (GB); Jason Ronald Fullman, Cheshire (GB); Helen Patricia Atkinson, Cheshire (GB); Emma Pawson, Cheshire (GB)

Correspondence Address: SALTER & MICHAELSON, THE HERITAGE BUILDING, 321 SOUTH MAIN STREET, PROVIDENCE, RI, 029037128

(21) Appl. No.: 10/873,089

(22) Filed: Jun. 22, 2004

Related U.S. Application Data
(63) Continuation of application No. 10/390,562, filed on Mar. 17, 2003, now abandoned.

(30) Foreign Application Priority Data
Mar. 18, 2002 (GB) 0206344.4

(51) Int. Cl. A61K 7/075; A61K 7/08

(52) U.S. Cl. 424/70.21

(57) ABSTRACT

A package of cleaning composition comprises a surfactant, an aqueous acidic component and an aqueous alkaline component, the acid and alkali being provided in separate compartments within the package. On dispensing from the package the acid and alkali react together creating carbon dioxide gas which acts upon the surfactant to create a lather. The acid and alkali components both have a viscosity and less than 4000 cps.

This invention relates to a cleaning composition and more particularly, but not exclusively to a personal cleaning composition intended for use in a shower.

Although personal cleaning compositions have been particularly developed for showers, such as shower gels, a significantly large number of people apparently prefer to use a conventional bar of soap in the shower rather than a shower gel. It is believed that one factor responsible for resistance to use of shower gels is connected with lather generation. Shower gels are provided in containers for dispensers from which the user must obtain a dose. This finite amount of gel will produce a finite quantity of lather, but in order to produce sufficient lather the user must apply shear to the gel for example by rubbing it on a part of the body. However, the lather so produced is soon washed away before the user is able to apply it to a part of the body and create a lather on the body. A bar of soap, on the other hand, provides a continuous supply of lather even if the stream of water from the shower head is directed onto the soap bar.

It is of course known that instantaneous lather can be obtained from cleaning compositions stored in aerosol containers. The release of such compositions from an aerosol together with a propellant creates foam ready for immediate use. However, aerosol-based compositions are expensive and furthermore carry the risk that the propellant gas is flammable.

EP 0745666A discloses an instantly foaming system without the use of an aerosol container. Here a two-phase aqueous system is employed whereby, when the two phases are combined together, a gas is generated which acts upon the surfactant in the system to produce lather. Typically, the reactive component in one phase is citric acid and in the other is sodium bicarbonate. However, in-use, the lather provided from washing with the disclosed example in EP 0745665A is quite sticky, stringy and slimy in nature. This would be a major barrier to consumer acceptance. Further work carried out on this system showed tat where the surfactant blend comprised primarily standard personal cleaning surfactants (i.e. a high foaming anionic surfactant was used as the primary surfactant) and either of the two separate phases had a viscosity of more than 4000 cps (as measured on a Brookfield Viscometer model number RDVI+ at 20° C., spindle 4, speed 20), this unacceptable lather persisted.

The present invention has been made from a consideration of this problem.

According to the present invention there is provided a cleaning composition provided in a package, wherein the cleaning composition comprises at least one surfactant, a first aqueous acidic component and a second aqueous alkaline component, the first and second components being located in separate compartments within the package prior to being dispensed therefrom, whereby when the first and second components are combined together, before or after dispensing, carbon dioxide gas is generated which acts on the surfactant to create a lather, characterised in that both the first and second components of the composition have a viscosity of less than 4,000 cps (measured on a Brookfield Viscometer model number RDVI+ at 20° C. spindle 4 speed 20).

This may be achieved either by the surfactants consisting primarily of one or more non-ionic surfactants and/or one or more amphoteric surfactants or by incorporating into the composition of the present invention a carefully balanced combination of at least one surfactant and at least one electrolyte.

Electrolytes are added to the present Invention for example to produce the carbon dioxide gas or optionally to buffer the acid. However, ionic surfactants can be affected by electrolytes such that where ionic surfactants are used as the primary surfactant the viscosity of the composition can undesirably increase to above 4000 cps.

Electrolyte addition to an ionic surfactant solution increases the ionic strength of the water. This has a dual effect. Firstly, it reduces the electrostatic repulsion between the like-charged head groups. This allows the ionic surfactant molecules to pack more closely together and as a consequence micelles and micelle structures are formed at lower concentrations. As the ionic strength of the water is increased larger micelle networks are formed and this gives rise to an increase in product viscosity e.g. over 4000 cps. The second effect of increasing the ionic strength of the solution is that the life-span of the micelles is reduced. Micelles are constantly breaking down and reforming and, when a certain ionic strength is reached, the lifetime of the micelle can become shorter than the time it takes to reform. This leads to a disruption of the micelle network and product viscosity dramatically decreases e.g. below 4000 cps. Hence as electrolyte levels are increased in an ionic surfactant solution, the viscosity of the product builds to a maximum and then is dramatically decreased as these processes occur. Consumer acceptable foam can therefore be produced either before this viscosity build above 4000 cps occurs or once this viscosity build has been disrupted back to below 4000 cps. This can be seen in the illustration in FIG. 4.

Therefore, the compositions preferably comprise non-ionic and/or amphoteric surfactants as the inclusion of an electrolyte in such compositions does not adversely affect the viscosity and therefore the foam characteristics of the composition. However, ionic surfactant/electrolyte combinations may be selected which also provide a composition having the desired viscosity and foam characteristics such that there is a disruption of the micelle network and the viscosity drops below 4000 cps, measured as above. Ionic surfactant/electrolyte combinations may be used alone or in conjunction with at least one other type of surfactant.

Preferably both the first and second components of the composition have a viscosity of less than 2000 cps, measured as above.

The preferred surfactant of the composition comprises one or more of the following either alone or in combination: alkyl polyglucoside (APG) (non-ionic) and cocamidopropyl betaine (amphoteric). Any non-ionic or amphoteric surfactants would be suitable but alkyl polyglucosides are noted for their exceptionally high foaming performance. Other examples of amphoteric surfactants which could be used include amphoacetates (e.g. cocoamphoacetate) and amphopropionates (e.g. sodium cocoamphopropionate). Other examples of non-ionic surfactants which could be used include fatty alcohol ethoxylates (e.g. laureth-4), fatty acid alkanolamides (e.g. cocamide DEA) and amine oxides (e.g. alkyl dimethyl amine oxide). This list is not exhaustive and does not preclude the use of other amphoteric and non-ionic surfactants as well as anionic (such as sodium lauryl ether sulphate) and cationic surfactants.

The surfactant shall preferably constitute from 1 to 30% by weight of the total composition and more preferably from 5 to 20% by weight of the total composition.

Where the surfactant is a mixture of amphoteric and non-ionic surfactants, the amphoteric surfactant preferably constitutes from 5 to 95 by weight of the total amount of surfactant the remainder of the surfactant is said non-ionic surfactant(s).

Where the composition comprises an ionic surfactant and an electrolyte the ionic surfactant preferably constitutes from 0.1 to 10 by weight of the total composition. In addition to an ionic surfactant the composition may also comprise from 1 to 30 by weight of an amphoteric surfactant and/or a non-ionic surfactant.

That said, the amount of surfactant is chosen having regard to the intended purpose of the cleaning composition. Where the composition is intended to be a shower gel it shall comprise from 2.5 to 30% by weight of at least one surfactant.

Where the composition is intended to be a shaving cream it shall preferably comprise from 1 to 30% by weight of at least one surfactant.

Where the composition is intended to be a fabric cleaner it shall preferably comprise from 1 to 40% by weight of at least one surfactant.

The surfactant can be included with one or other component or may be kept separate and combined with the mixture of the two components when they are dispensed.

The level of electrolyte in each composition is preferably at least 1% by weight of the total and more preferably at least 5% by weight of the total and is present preferably in the acid component as sodium citrate and in the alkaline component as potassium bicarbonate. As referred to previously the electrolyte may also contribute to the production of carbon dioxide gas or as a buffering system.

Suitable electrolytes are substances, the molecules of which may dissociate in solution to charged particles (ions). For example, potassium citrate, sodium bicarbonate, sodium carbonate, potassium carbonate, sodium sesquicarbonate and potassium sesquicarbonate.

The first acidic component further preferably comprises a buffer such that the pH of the first component is in the range from 2 to 6.5 and is preferably in the range from 3 to 6.

The use of the buffering system in the acid phase limits the potential for skin irritation by bringing the pH of the first component from 1.9 up to 3.6. The pH of the first component is ideally in the range from 4 to 5. This pH is much more acceptable for the skin, if the acid component is dispensed separately or in excess.

The first component preferably but not exclusively comprises an alpha hydroxy acid in combination with a salt thereof. Most preferred is citric acid in combination with a citrate, preferably sodium citrate. The second component comprises a carbonate or bicarbonate. Although sodium carbonate or sodium bicarbonate may be used, ideally the second component comprises potassium bicarbonate. Potassium bicarbonate is over twice as soluble as sodium bicarbonate. This allows much higher levels to be used and therefore allows a much higher amount of gas to be generated which means that foam is generated in higher quantities and can be of higher viscosity. As a key consumer driver for products of this type is the amount and speed of lather production the use of potassium bicarbonate yields significant advantages.

The composition of the invention may include other additives which are usually included in cleaning compositions, for example any of the following either alone or in combination: thickeners, fragrance and the like.

The invention is not restricted to facial cleansers or shower gels. Compositions according to the invention may be used for other cleaning uses including shaving creams, hand washes shampoos and possibly fabric cleaners or hard surface cleaners.

### EXAMPLES

### Example 1

Two components of a light foaming facial cleanser were formulated as follows:

Component A

| INGREDIENTS | % w/w |
|---|---|
| Deionised Water | to 100% |
| Citric Acid Monohydrate | 7.0% |
| Trisodium Citrate | 16.5% |
| Decyl Glucoside | 2.5% |
| Cocamidopropyl Betaine | 2.5% |
| Preservative | q.s. |
| Colour | q.s. |
| Fragrance | q.s. |

Component B

| INGREDIENTS | % w/w |
|---|---|
| Deionised Water | to 100% |
| Potassium Bicarbonate | 20.0% |
| Decyl Glucoside | 2.5% |
| Cocamidopropyl Betaine | 2.5% |
| Preservative | q.s. |
| Colour | q.s. |
| Fragrance | q.s. |

### Example 2

Two components of an instant lathering shower gel were formulated as follows:

Component A

| INGREDIENTS | % w/w |
|---|---|
| Deionised Water | to 100% |
| Citric Acid Monohydrate | 7.0% |
| Trisodium Citrate | 16.0% |
| Lauryl Glucoside | 10.0% |
| Cocamidopropyl Betaine | 10.0% |
| Preservative | q.s. |
| Colour | q.s. |
| Fragrance | q.s. |

Component B

| INGREDIENTS | % w/w |
|---|---|
| Deionised Water | to 100% |
| Potassium Bicarbonate | 20.0% |
| Lauryl Glucoside | 10.0% |
| Cocamidopropyl Betaine | 10.0% |
| Preservative | q.s. |
| Colour | q.s. |
| Fragrance | q.s. |

### Example 3

Component A

| INGREDIENTS | % w/w |
|---|---|
| Deionised Water | to 100% |
| Cocamidopropyl Botaine | 15.0% |
| Citric Acid Monohydrate | 7.0% |
| Trisodium Citrate | 16.8% |
| Solubilisemside | 1.00% |
| Fragrance | q.s. |
| Colour | q.s. |
| Preservatic | q.s. |

Component B

| INGREDIENTS | % w/w |
|---|---|
| Deionised Water | to 100% |
| Decyl Glucoside | 15.0% |
| Potassium Bicarbonate | 15.0% |
| Solubiliser | 1.0% |
| Preservative | q.s. |
| Fragrance | q.s. |
| Colour | q.s. |

### Example 4

Component A

| INGREDIENTS | % w/w |
|---|---|
| Deionised Water | to 100% |
| Cocamidopropyl Betaine | 15% |
| Citric Acid Monohydrate | 7.0% |
| Trisodium Citrate | 17.0% |
| Solubiliser | 1.0% |
| Preservative | q.s. |
| Fragrance | q.s. |
| Thickener | q.s. |
| Colour | q.s. |

Component B

| INGREDIENTS | % w/w |
|---|---|
| Deionised Water | to 100% |
| Decyl Glucoside | 15% |
| Potassium Bicarbonate | 15.00% |
| Solubiliser | 10.0% |
| Preservative | q.s. |
| Fragrance | q.s. |
| Thickener | q.s. |
| Colour | q.s. |

### Example 5

Component A

| INGREDIENTS | % w/w |
|---|---|
| Deionised Water | to 100% |
| Sodium Laureth Sulfate | 10.0% |
| Cocamidopropyl Betaine | 2.5% |
| Citric Acid Monohydrate | 7.0% |
| Trisodium Citrate | 17.0% |
| Solubiliser | 1.0% |
| Preservative | q.s. |
| Fragrance | q.s. |
| Thickener | q.s. |
| Colour | q.s. |

Component B

| INGREDIENTS | % w/w |
|---|---|
| Deionised Water | to 100% |
| Decyl Glucoside | 15% |
| Potassium Bicarbonate | 15.00% |
| Solubiliser | 1.0% |
| Preservative | q.s. |
| Fragrance | q.s. |
| Thickener | q.s. |
| Colour | q.s. |

The present invention will now be described further by way of example only and with reference to the following drawings in which:

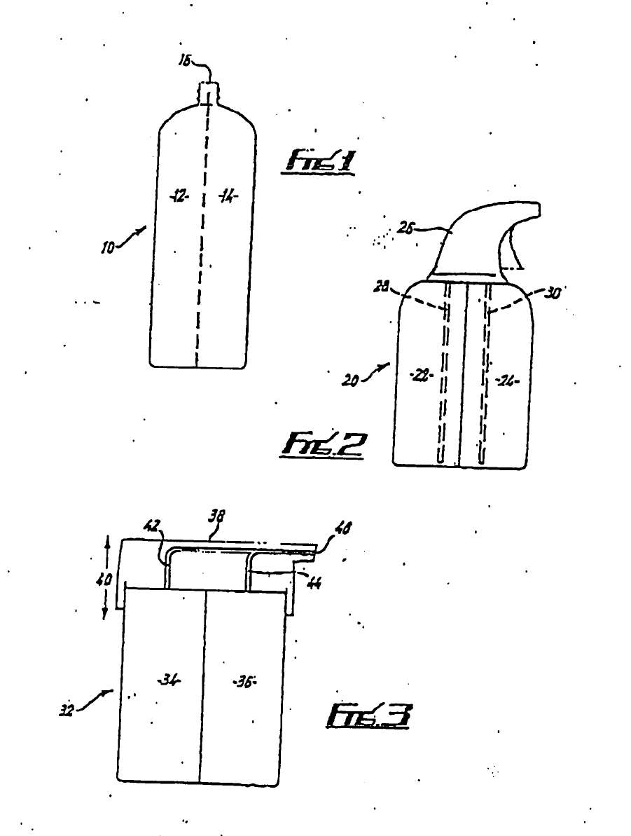

FIG. 1 is a diagrammatic side view of one type of package suitable for use with the cleaning composition described herein;

FIG. 2 is a diagrammatic side view of a second type of package suitable for use with the cleaning composition described herein;

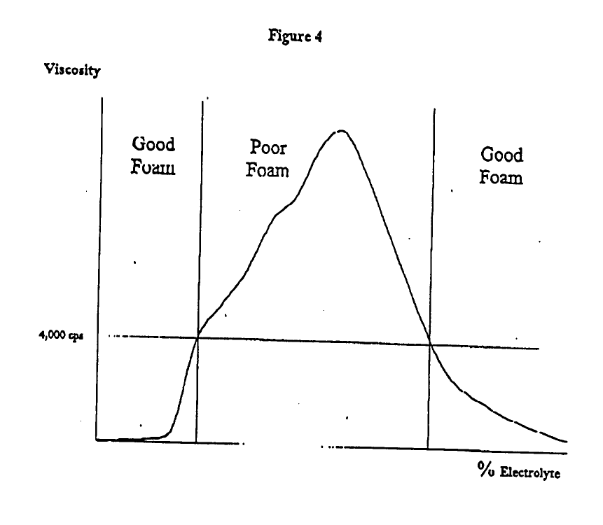

FIG. 3 is a diagrammatic side view of a third type of package suitable for use with the cleaning composition described herein; and

FIG. 4 is a graph showing the relationship between the concentration of electrolyte in the composition and the viscosity of the composition.

In each example the components were charged into separate compartments 12, 14, within a flexible container 10 illustrated in FIG. 1. Each compartment has an outlet leading to a common nozzle 16 which can be closed by a cap not shown. When the container was compressed the contents of both compartments were dispensed through the nozzle. A reaction between the acid and the bicarbonate immediately occurred releasing carbon dioxide gas which in turn created neutral, dense, creamy foam.

The invention is not restricted to the specific embodiments just described, nor is it restricted to facial cleaners or shower gels. Compositions according to the invention may be used for other cleaning uses including shaving creams, fabric cleaners and hard surface cleaners.

Other types of package can be used with the composition of the invention. For example as shown in FIG. 2 a container 20 having two compartments 22, 24 has a pump action spray 26 mounted on the top thereof with dip tubes 28, 30 extending into the compartments of the container. This kind of package is known for use in connection with a concentrated substance in one compartment and a dilutent in the other whereby the mixture that is dispensed is diluted concentrate.

Another type of package that can be used with the composition of the invention is shown in FIG. 3 and comprises a container 32 having two compartments 34, 36. A cap 38 reciprocally movable is indicated by arrows 40 which action pumps the contents of the compartments into outlets 42, 44. The compartment outlets merge together to form a common outlet 46. This type of package is also known but for keeping two ingredients of a cosmetic separate until they are dispensed for use in order to prevent one ingredient which is an emulsion being broken by the other ingredient.

The packages and examples described herein are by way of illustration only.

1. A cleaning composition provided in a package, wherein the cleaning composition comprises at least one surfactant, a first aqueous acidic component and a second aqueous alkaline component, the first and second components being located in separate compartments within the package prior to being dispensed therefrom, whereby when the first and second components are combined together, before or after dispensing, carbon dioxide gas is generated which acts on the surfactant to create a lather, characterised in that both the first and second components of the composition have a viscosity of less than 4000 cps.

2. A composition according to claim 1, wherein the first component has a viscosity of less than 2000 cps.

3. A composition according to claim 1, wherein the second component has a viscosity of less than 2000 cps.

4. A composition according to claim 1 wherein the surfactant comprises any of the following either alone or in combination, a non-ionic surfactant and an amphoteric surfactant.

5. A composition according to claim 4, wherein the amphoteric surfactant comprises any of the following either alone or in combination, amphoacetates and amphopriopionates, betaines.

6. A composition according to claim 4, wherein the non-ionic surfactant comprises any of the following either alone or in combination, alkyl polyglucoside, fatty alcohol ethoxylates, fatty acid alkanolamides and amine oxides.

7. A composition according to claim 1, wherein the amphoteric surfactant constitutes from 5 to 95% by weight of the total amount of surfactant.

8. A composition according to claim 1, wherein the surfactant comprises an ionic surfactant together with at least one electrolyte.

9. A composition according to claim 8, wherein the surfactant further comprises an amphoteric surfactant and/or a non-ionic surfactant.

10. A composition according to claim 1, wherein the electrolyte constitutes at least 1% by weight of the total composition.

11. A composition according to claim 9, wherein the electrolyte constitutes at least 5% by weight of the total composition.

12. A composition according to claim 1, wherein said surfactant constitutes from 1% to 30% by weight of the total composition.

13. A composition according to claim 1, wherein the first acidic component has a pH in the range from 2 to 6.5.

14. A composition according to claim 9, wherein the pH of the first acidic component is in the range from 3 to 6.

15. A composition according to claim 1, wherein the first acidic component comprises an alpha hydroxy acid in combination with a salt thereof.

16. A composition according to claim 1, wherein the second alkaline component comprises a carbonate or bicarbonate.

17. A composition as referred hereinbefore and with reference to any examples of 1 to 5.
